(19) United States
(12) Patent Application Publication
(10) Pub. No.: US 2005/0042170 A1
(43) Pub. Date: Feb. 24, 2005

(54) Method and device for generating mists and medical uses thereof

(75) Inventors: Yandong Jiang, Newton, MA (US); Massimo Ferrigno, Brookline, MA (US)

Correspondence Address: Michael A. Sanzo;Fitch, Even, Tabin & Flannery, 1801 K Street, N.W., Suite 401L, Washington, DC, 20006-1201, US

(73) Assignee: The Brigham and Women's Hospital, Inc.

(21) Appl. No.: 10/776,642

(22) Filed: Feb. 12, 2004

Related U.S. Application Data
(60) Provisional application No. 60/447,335, filed on Feb. 14, 2003.

(51) Int. Cl. A61L 9/04; A61M 11/00; B65D 83/06; B65D 83/14

(52) U.S. Cl. 424/45; 128/200.23; 222/400.5

(57) ABSTRACT

The present invention is directed to a method and device for generating a mist. Mist is created by a sudden drop in the pressure of a gas/liquid mixture. This results in the rapid expansion of the volume of the gas component and the consequent breaking of the liquid into tiny droplets. The method and device allow for the precise control of both the size of the liquid droplets and the density of the mist produced. The mists may be used in a wide variety of applications, including medical procedures in which the body temperature of a patient is rapidly changed and any industrial procedure where a mist is needed.

### CROSS REFERENCE TO RELATED APPLICATION

This application claims priority to U.S. provisional application no. 60/447,335, filed on Feb. 14, 2003, which is incorporated in its entirety herein by reference.

### FIELD OF THE INVENTION

The present invention is directed to a method and device for generating mists. Using this method and device, the size of the droplets in the mist, the output of the mist and the density of the mist can be precisely controlled. The mists can be used in a wide variety of applications but are particularly well suited to medical procedures in which the temperature of a patient must be rapidly changed.

### BACKGROUND OF THE INVENTION

The ability to rapidly change the body temperature of a patient has a number of important medical applications. Hypothermia is induced by physicians to protect the heart and brain of patients during cardiac surgery or operations involving cerebral blood vessels. Physicians may also rapidly cool a patient's body to protect brain tissue following traumatic injury, during resuscitation from cardiac arrest and to help prevent brain damage after a stroke. In other instances, the rapid warming of a patient can be important, e.g., in cases where hypothermia has resulted from an accident.

One approach that may be taken to altering body temperature is through the inhalation of gases or liquids (U.S. Pat. No. 6,303,156; Forman, et al., J. Surg. Res. 40:36-42 (1986)). Unfortunately, these procedures have met with limited success because of the relatively poor ability of gases to transfer heat and respiratory difficulties caused by the high viscosity of liquids. Mists, which combine the relatively low viscosity of gases with the high heat transfer capacity of liquids, appear to offer the best medium for respiratory heat transfer. However, in order to be optimally effective, a means must be available for generating a mist with a high concentration of small diameter liquid droplets.

At present, there are two methods that are commonly used to produce a mist from a liquid using gas as a carrier (O'Callaghan, et al., Thorax 52:S31-44 (1997)). One method uses a jet nebulizer in which a compressed gas, typically air, is released through a small hole. Rapid expansion of the gas generates a negative pressure (Venturi effect) which draws liquid into a feeding tube system where it is atomized. Larger droplets adhere to baffles along the walls of the nebulizer device and small aerosol droplets are released continuously from the nebulizer chamber. Unfortunately, conventional jet nebulizers are highly inefficient. Between 93% and 99% of the droplets produced are caught on the internal baffles, resulting in a low output. In addition, the homogeneity of the droplets produced by a jet nebulizer is generally poor. Adding a filter improves homogeneity but further reduces output and alternative designs have been proposed in an attempt to overcome these problems (Nerbrink, et al., J Aerosol Med. 7:259-276 (1994)). However, even with alternative designs, the efficiency of mist output is limited.

The second commonly used method for producing mists utilizes ultrasonic nebulizers. These devices use a rapidly vibrating piezoelectric crystal to produce aerosols. Vibrations from the crystal are transmitted to the surface of the liquid where standing waves are formed. Droplets break free from the crests of these waves and are released as a mist. As with jet nebulizers, ultrasonic nebulizers are usually very inefficient and, in addition, tend to cause complex molecules, e.g., drugs, to break down.

### SUMMARY OF THE INVENTION

The present invention is based upon the development of a method and device which are capable of generating mists with a high concentration of droplets of very small diameter. The various components of the device can best be understood by reference to FIGS. 1-6. The mist generated may be used for a wide variety of purposes, but is particularly well suited to medical procedures designed to rapidly elevate or lower a patient's body temperature by having them inhale mists of a controlled temperature.

In its first aspect, the invention is directed to a mist-producing device in which there is a high pressure pump (29) connected to a gas/liquid container (39). The pump has a chamber (6) which is connected to a high pressure compartment (30) located within the gas/liquid container. The pump chamber is also connected by a gas pipe (11) to a gas reservoir (24) and by a liquid pipe (10) to a liquid reservoir (13). In addition to having a high pressure compartment, the gas/liquid container also has a low pressure compartment (40) (typically at a pressure at least one atmosphere lower than in the high pressure compartment).

Within the high pressure compartment (30) of the gas/liquid container (39), there are one or more orifices (20) which, when fully open, are 1-25 micrometers in diameter. The orifices connect the high pressure compartment (30) to the low pressure compartment (40) of the gas/liquid container (39). The low pressure compartment (40) is connected to a mist pipe (22) that leads to means for releasing mist from the low pressure compartment to outside the device. These means can vary greatly depending upon the intended use for the device. For example, when used in medical procedures, the mist may be released by a valve into an endotracheal tube of the patient through a ventilator (43), if the patient is intubated. It can also be delivered through a respiratory mask designed to cover a patient's nose and mouth, if a patient is breathing spontaneously. When used in a non-medical setting, mist may be released by a nozzle or spray gun.

In preferred embodiments, the orifices (20) of the device are formed by heating elements (31) that are interposed between the high pressure compartment (30) and low pressure compartment (40) of the gas/liquid container (39). The purpose of the heating elements is to control the temperature of the mist droplets as they are formed. The high pressure compartment (30) of the device may also have a sensor (15) designed to respond to the level of liquid within the chamber and means for mixing the contents of the high pressure compartment to ensure homogeneity. As shown in FIG. 2, one way for accomplishing mixing is by a motor-driven rotating blade (18). However, other commonly used devices for mixing may also be employed. The mist-generating device preferably includes a valve on the liquid pipe (10) leading from the liquid reservoir (13) to the pump chamber (6), and on the gas pipe (11) leading from the gas reservoir (24) to the pump chamber (6). The opening and closing of these valves should be under the control of the level sensor (15) such that, when the liquid level in the high pressure compartment is lower than the sensor, the valve on the liquid pipe is open and the valve on the gas pipe is closed. In contrast, when the liquid level in the high pressure compartment is at or above the level sensor, the valve on the liquid pipe should be closed and the valve on the gas pipe should be open.

In additional preferred embodiments, the device described above has a pressure gauge (16) connected to the high pressure compartment (30). This should be connected to a motor driving the high pressure pump such that the pump is turned off when a pre-selected pressure is reached and is on at other times. In the embodiment shown in FIG. 2, the high pressure pump is a piston pump and is separated from the high pressure compartment (30) by a diaphragm (28). The diaphragm is designed to keep pump components such as oil from entering gas/liquid mixtures within the high pressure compartment. The low pressure compartment (40) of the gas/liquid container (39) may be connected to the liquid reservoir (13) by a liquid drain pipe (26) designed to return condensation found within the low pressure compartment to the reservoir.

The device described preferably includes a balloon reservoir (38) connected to the gas pipeline (11). This is designed to prevent negative pressure from forming within the gas line. As safety features, both the high pressure compartment (30) and the low pressure compartment (40) may contain pop off valves. The pop off valve in the lower pressure compartment is particularly important in cases where the device is used for medical applications in that it ensures that patients are not exposed to potentially harmful high pressures. The device may also have a switch (32) designed to open and close the orifices (20) leading from the high pressure compartment (30) to the low pressure compartment (40). Ideally, this switch should be capable of closing all, or some, of the orifices. The switch (32) can be controlled precisely and slides up and down relative to the orifice housing (31). This allows the opening (41) in the switch (32) to overlap with the opening of the orifice housing (31) and to change the cross sectional area of the stream (42).

The invention is also directed to methods for generating mist from a liquid by loading the liquid into the devices described above and then releasing mist from the low pressure compartment of the gas/liquid container. By generating compositions in which there is a high concentration of droplets of very small diameter, the device is ideally suited for humidifying, applying water or chemicals to crops in agricultural settings, to industrial applications such as spray painting, and to any other procedure which requires the generation of a mist.

One particularly important use of the devices is for either rapidly increasing or decreasing the body temperature of a patient. Mists administered to patients by ventilation should be comprised of a mixture of a physiologically acceptable gas (preferably air or oxygen) and a physiologically acceptable liquid (preferably saline). Depending upon the objective of the procedure, the mist may be administered either below the body temperature of the patient (for example, at 1° C.-30° C.) or at a temperature above the patient's body temperature (e.g., at 37° C.-42° C.).

The average size of the liquid droplets within a mist will depend upon the pressure gradient at which gas/liquid mixtures are extruded through orifices, the size of the orifices and other factors. The average size of the particles should be no more than 5 microns in diameter, preferably no more than 2 microns in diameter, and still more preferably, less than 1 micron in diameter. The ratio of gas to liquid within mists can vary over a wide range but, in medical applications, the gas should generally constitute at least 90% of the mist by volume and preferably at least 95% by volume. Similarly, the liquid should generally constitute no more than 10% of the mist by volume and preferably no more than 5%. Methods in which the body temperature of a patient is reduced will be useful in preparing for cardiac or neurosurgery, in treating a patient in hemorrhagic shock and to prevent brain damage subsequent to a stroke or after cardiac resuscitation. Methods in which the body temperature of a patient is increased will be useful as a treatment for hypothermia.

### BRIEF DESCRIPTION OF THE DRAWINGS

The mist generating device of the present invention is illustrated in FIGS. 1-6. The main components shown in the drawings are as follows:
1: motor-driven wheel for driving the piston (5) of the high pressure pump;
2: lever attached to piston (5) and motorized wheel (1);
3: pump oil inside pump housing (4);
4: pump housing;
5: piston attached to lever (2) and sliding within pump chamber (6);
6: pump chamber;
7: one-way valve permitting gas and liquid to enter high pressure compartment (30) of gas/liquid container (39);
8: one-way valve permitting gas flow from gas reservoir (24) to pump chamber (6);
9: one-way valve permitting liquid flow from liquid reservoir (13) to pump chamber (6);
10: liquid pipe connecting liquid reservoir (13) with pump chamber (6);
11: gas pipe connecting gas reservoir (24) to pump chamber (6);
12: liquid within liquid reservoir (13);
13: liquid reservoir;
14: feeder for introducing liquid into liquid reservoir (13);
15: level sensor for sensing liquid level in high pressure compartment (30), interacting with two switches (33 and 34);
16: pressure gauge measuring pressure in high pressure compartment (30);
17: gas/liquid mixture inside high pressure compartment (30);
18: motor driven stir blade for mixing contents of high pressure compartment (30);
19: high pressure pop off valve;
20: orifices for releasing gas/liquid mixture from high pressure compartment (30) to low pressure compartment (40);
21: mist formed after extrusion of gas/liquid mixture from high pressure compartment (30) to low pressure compartment (40);
22: mist pipe leading to release interface which may be a nozzle, spray gun or respiratory mask;
23: liquid condensate formed from mist in low pressure compartment (40);
24: gas reservoir connected to pump chamber (6) by gas pipe (11);
25: pressure gauge measuring pressure in gas pipe (11);
26: liquid drain pipe connecting low pressure compartment (40) with liquid reservoir (13);
27: pump;
28: diaphragm separating high pressure pump chamber (6) from high pressure compartment (30);
29: high pressure pump;
30: high pressure compartment in gas/liquid container (39);
31: orifice housing with walls made up of heating elements and attached to the gas/liquid container; the heating elements heat the gas/liquid mixture as it is extruded from high pressure compartment (30) to low pressure compartment (40);
32: switch for opening and closing orifices (20);
33: switch connected to level sensor (15) and to one-way valve (9) on liquid pipe (10);
34: switch connected to level sensor (15) and controlling valve (8) on gas pipe (11);
35: low pressure pop off valve;
36: switch connecting liquid feed (14) to liquid reservoir (13) and under control of liquid sensor (37);
37: liquid sensor in liquid reservoir (13);
38: balloon gas reservoir preventing negative pressure from forming in gas pipe (11);
39: gas/liquid container;
40: low pressure compartment of gas/liquid container (39);
41: opening on orifice switch (32);
42: opening formed by the overlap between the opening (41) in orifice switch (32) and the opening (20) in the orifice housing;
43: respiratory circuit and ventilator;
44: high pressure jet of mist released from the high pressure compartment;
45: ultrasonic nebulizer;
46: hydrophobic coating on the surface of the ultrasonic nebulizer;
47: homogeneous mist reflected from the coated surface of the ultrasonic nebulizer; and
48: alternator controlling vibration frequency of ultrasonic nebulizer.

FIG. 1: FIG. 1 shows an overview of the mist generating device of the present invention. There are essentially two major components: a high pressure pump (29) and a gas/liquid container (39) with both a high pressure compartment (30) and a low pressure compartment (40).

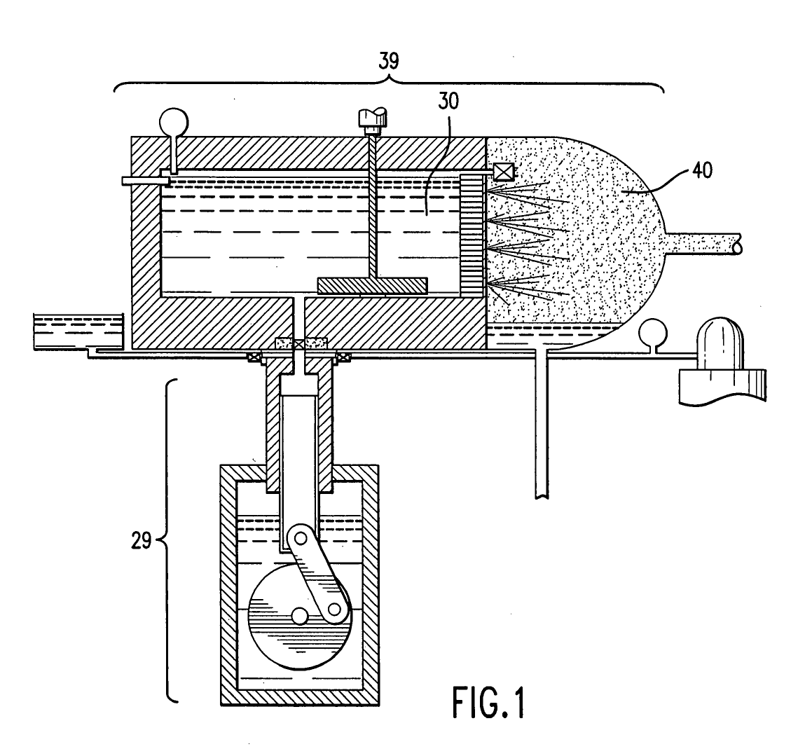

FIG. 2: FIG. 2 is a detailed drawing showing the main components of the mist generating device of the present invention. The lower part of the figure shows a high pressure piston pump in which a motor driven wheel (1) and lever (2) drives a piston (5). These components are located within a pump housing (4) filled with oil (3). Pump oil is prevented from entering the pump chamber (6) by a diaphragm (28). There is a one-way valve (7) leading from the pump chamber to the high pressure compartment of a gas/liquid container (39). The device includes a gas reservoir (24) which leads into the pump chamber (6) through a gas pipe (11). Located along this pipe is a pressure gauge (25), a balloon reservoir (38) and a one-way valve (8) which is under the control of a switch (34). There is also a liquid reservoir (13) that is connected to the pump chamber (6) by a liquid pipe (10). This pipe also has a one-way valve (9) under the control of a switch (33). The gas and liquid are pumped into a high pressure compartment where they form a gas/liquid mixture (17). The high pressure compartment includes a pressure gauge (16), a motor driven mixing blade (18) and a level sensor (15) which, depending upon the amount of liquid in the high pressure compartment, opens or closes the switch (33) on the liquid pipe (10) and the switch (34) on the gas pipe (11). There is also a pop off valve (19) designed to release pressure within the high pressure compartment if it becomes too high. The high pressure compartment has orifices (20) of very small diameter (1-25 micrometers) which can be opened or closed. When open, the orifices permit the extrusion of the gas/liquid mixture from the high pressure compartment to a low pressure compartment (40) also within the gas/liquid container. During the extrusion process, gas quickly expands and liquid is broken into very small diameter particles, thereby forming a mist (21). The mist is carried along a mist pipe (22) which leads to an interface where it is released to outside of the device. The low pressure compartment also includes a low pressure pop off valve (35) designed to release pressure if it becomes excessive. A certain amount of condensate (23) forms from the mist (21) in the low pressure compartment (40) and is transported by means of a pump (27) along a liquid drain pipe (26) to the liquid reservoir (13). The liquid reservoir has a level sensor (37) which measures the amount of fluid (12) present within the reservoir. The level sensor controls the opening and closing of a switch (36) controlling the amount of liquid introduced into the reservoir through a feeder (14).

FIG. 3: FIG. 3 is an enlarged schematic drawing of the orifices (20) leading from the high pressure compartment to the low pressure compartment of the gas/liquid container. The orifices are formed by heating elements (31) which regulate the temperature of the gas/liquid mixture being extruded. The opening and closing of orifices is under the control of a switch (32). Upon passing through orifices, the gas/liquid mixture (17) in the high pressure compartment is broken into small particles due to the rapid expansion of gas and forms a mist (21). Also shown in FIG. 3 is a high pressure pop off valve (19) which releases pressure within the high pressure compartment if it becomes excessive.

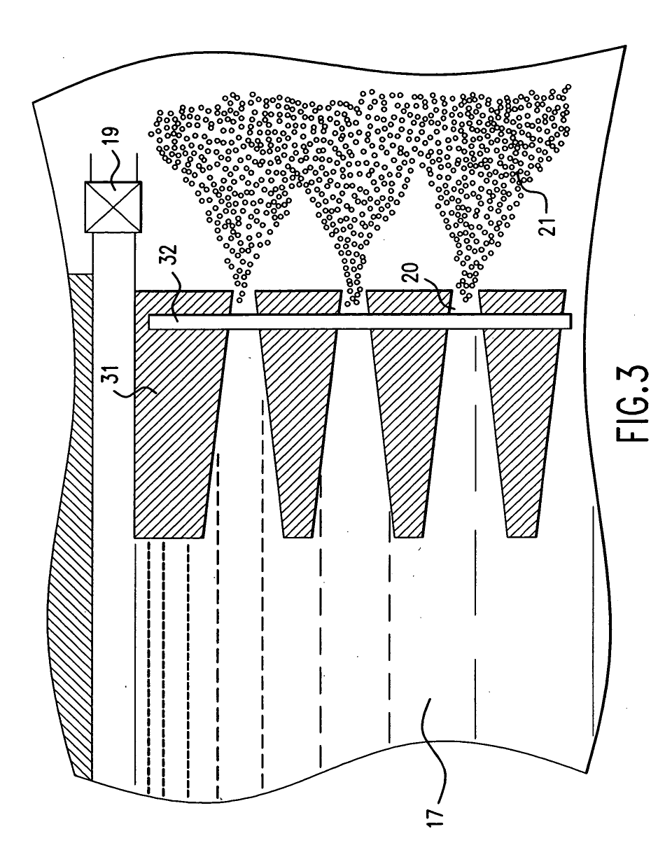

FIG. 4: FIG. 4 is a close up view of the mist pipe (22) leading to a release interface which, in this case, is a respiratory circuit and ventilator (43). In the figure, the patient is shown as having been intubated. In alternative designs, mist may be released into a respiratory mask that covers a patient's mouth and nose or, in nonmedical applications, the mist may be released by a nozzle or spray gun. Also shown in the figure is a low pressure pop off valve (35).

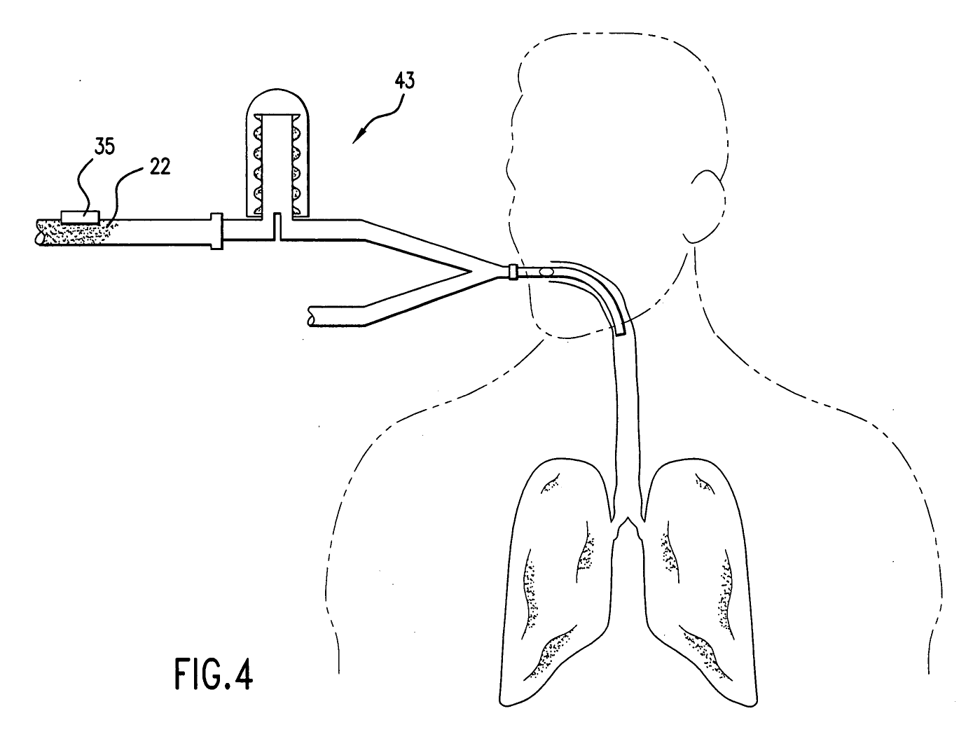

FIG. 5: FIG. 5 is a close up view of the switch (32) for opening and closing orifices (20). Opening and closing is accomplished by sliding the plate shown in the picture so that its opening (41) either corresponds to or is displaced from the corresponding opening in the orifice housing (20).

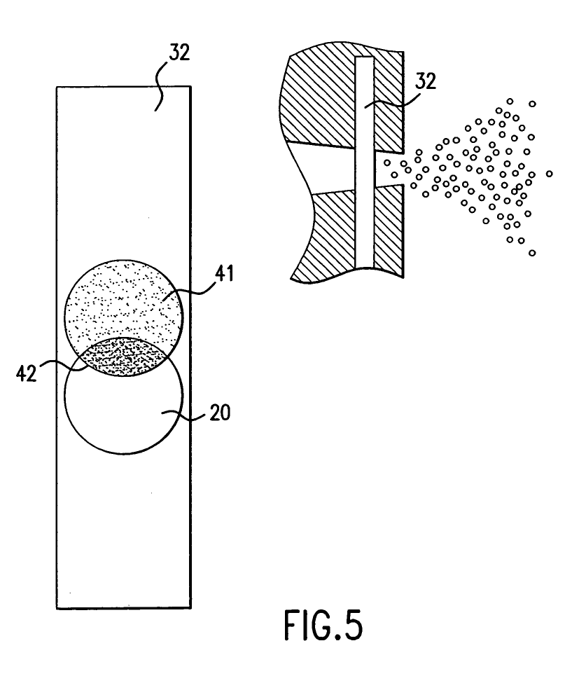

FIG. 6: FIG. 6 shows an arrangement in which the surface of an ultrasonic nebulizer (46) is positioned in front of an orifice (20) of the high pressure compartment (30). A jet of high pressure mist (44) released from the orifice strikes the surface of the ultrasonic nebulizer which is vibrating at a frequency controlled by an alternator (48). The surface of the ultrasonic nebulizer is covered with a hydrophobic coating (46) to prevent liquid from freezing and mist striking this coating is deflected away (47). In effect, mist is generated at two different points, once from the device described in FIGS. 1-5 and once from the surface of the ultrasonic nebulizer. The first generation of mist produces particles of very small diameter and the second makes the particles more homogeneous in terms of size. The mist from the coated surface of the ultrasonic nebulizer may then be directed to a respiratory mask, pipe, nozzle or similar interface and released from the gas/liquid container.

### DETAILED DESCRIPTION OF THE INVENTION

The present invention is directed to a method for producing mists that uses an approach different from methods currently in use, i.e., jet nebulization and ultrasonic nebulization. In particular, mists are produced by a device that releases a mixture containing a pressurized gas and a pressurized liquid through an orifice with a diameter in the micrometer range. When the mixture escapes from the orifices, the decompressed gas rapidly expands its volume up to 1244 times and breaks the liquid stream escaping from the orifices into tiny droplets.

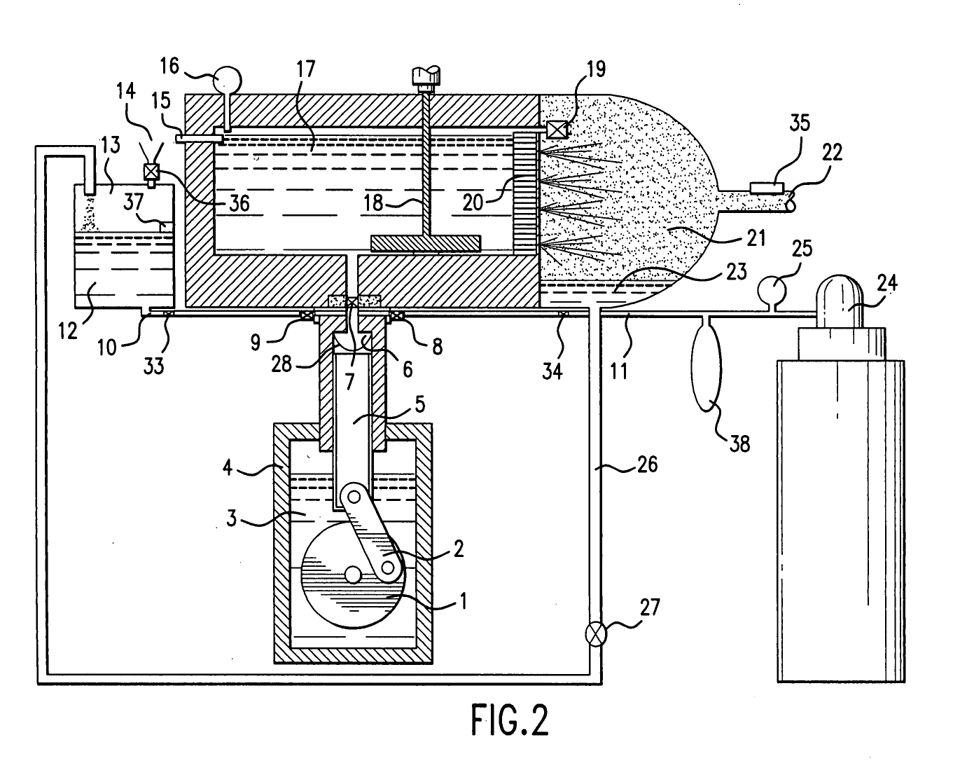

Pressure in the device is created by means of a high pressure pump which may take the form of any of the many types known in the art. In a preferred embodiment shown in FIG. 2, the pump has a motor that is connected to a wheel (I) that pushes a piston (5) by means of a lever (2). The housing (4) of the high pressure pump (29) holds oil (3) that lubricates the joints of the lever and piston. A diaphragm (28) separates the piston (5) from the pump chamber (6) and prevents oil from contaminating the high pressure compartment (30). When the piston retracts, it generates a negative pressure in the pump chamber (6) and gas or liquid is drawn from either the gas pipe (11) or the liquid pipe (10) through one-way valves (8) and (9). When the piston moves forward, it pushes the liquid or gas through the one-way valve (7) into the high pressure compartment (30) of the gas/liquid container (39).

When the liquid level in the high pressure compartment reaches level sensor (15), a signal is sent to switch (33) on the liquid pipe (10) to close the one-way valve (9) and, as a result, only gas is pumped into the high pressure compartment. If the liquid level falls below the level sensor (15), switch (33) opens valve (9) and switch (34) on the gas pipe (11) shuts off one-way valve (8). Thus, only liquid is pumped into the high pressure container under these circumstances. The pump will keep running until the high pressure compartment (30) reaches a target pressure as measured by a pressure gauge (16). At that point, the gauge will signal the pump to stop operation. As a safety feature, the high pressure compartment (30) includes a pop off valve (19) that releases pressure if it becomes unacceptably high.

The gas and liquid mixture within the high pressure compartment is continually mixed to prevent these components from separating from each other. This may be accomplished by any means known in the art, but one preferred method shown in FIG. 2 is by means of an electronically operated mixing blade (18).

In the high pressure compartment (30), the mixture (17) of gas and liquid is pressurized from about 2 atmospheres up to the critical pressure of the gas, at which point the gas component either is converted from its gas phase into its liquid phase or dissolves in the liquid in very high concentration. When this mixture is forced through orifices (20) into a low pressure compartment (40) of the gas/liquid container (39), there is a sudden reduction in pressure. This causes an expansion of the volume of the gas component of the mixture present in either gas or liquid phase. In contrast, the liquid component of the mixture will remain in liquid phase and its volume will not change appreciably despite the sudden reduction in pressure at the orifice. Nevertheless, the sudden expansion of the gas volume together with the high speed of the mixture being forced through the orifice breaks the liquid into tiny droplets, thereby forming a mist (21).

Due to the evaporation energy caused by the gas, the temperature of the mixture at the orifice will be reduced and the liquid component may be released either as cold droplets or cold solid particles, i.e., ice crystals. In order to prevent liquid from freezing at the orifices and to control the temperature of the mist generated, the orifice walls (31) are heating elements that maintain a desired temperature, i.e., 1° C.

The output (volume per unit of time) of mist produced can be controlled by means of a switch (32) that changes the number of orifices in the open state without altering the pressure gradient at the orifices. The volume ratio of the mist (volume of liquid/volume of gas) can be controlled by altering the pressure in the high pressure compartment (30) (the higher the pressure, the denser the gas in the container, the larger the expansion at the orifices and the lower the volume ratio of the mist). The size of the droplets in the mist can be controlled by changing the diameters of the orifices and/or the pressure gradient between the high pressure compartment and low pressure compartment (the smaller the diameter of the orifices and/or the higher the pressure gradient, the smaller the size of the droplets). The mist is directed to an interface (not shown in the drawings) between the mist generator and the mist utilizing system (also not shown). There is a low pressure (e.g., 40 cm of water) pop off valve (35) upstream of the interface. This valve prevents potentially damaging high pressures from occurring in the downstream system (for example, a patient's airway).

Within the low pressure compartment of the gas/liquid container, liquid condensate will form due to the collision of droplets. This may be removed by means of a pump (27) through a pipe (26) and transported back to the liquid reservoir (13) for reuse. Liquid may be introduced into the liquid reservoir (13) from a feeder (14). If the liquid level in the reservoir is below that of a level sensor (37), an automatic switch may be activated, allowing liquid to be fed into the reservoir by gravity from an appropriate container (not shown in the drawing). When the liquid level is above the level sensor, the automatic switch will be turned off.

Gas may be fed into the system through a gas pipe (11) from a gas reservoir (24). A regulator (25) may be included on the gas pipe (11) to convert the high pressure gas released from the gas tank into a low pressure gas (for example, 40 cm of water). A balloon (38) maybe installed on the gas pipe as a reservoir to prevent negative pressure and an automatic switch (34) can be used to control whether the one-way valve on the gas pipe (8) is open.

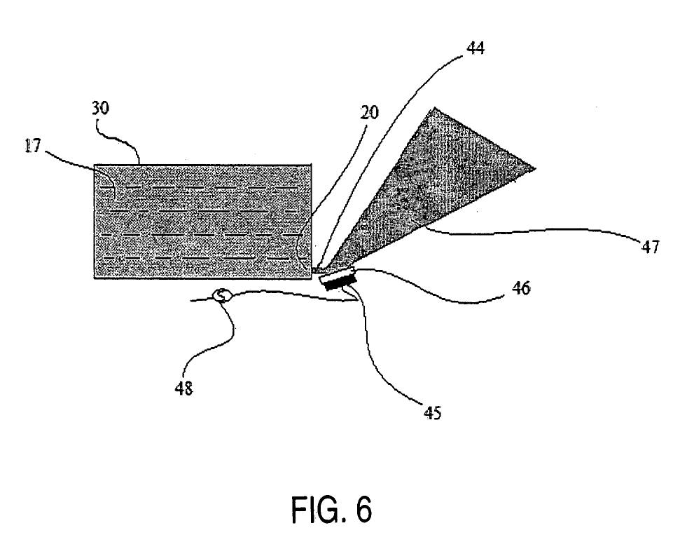

A standard ultrasonic nebulizer may be used to improve the uniformity of the size of the droplets produced by the device discussed above. FIG. 6 is a stylized drawing showing one way in which this can be done. The high pressure compartment is represented in the drawing as (30) and contains a gas/liquid mixture (17). The surface of the ultrasonic nebulizer is placed at an orifice (20) where a high-pressure jet (44) of mist is released from the high pressure compartment (30). In order to avoid formation of ice on the surface of the ultrasonic nebulizer (45), the surface is coated with a hydrophobic material (46). The nebulizer (45) and the coat (46) vibrate at the same frequency (for example 2.4 MHz) and produce a homogeneous mist (47) with, for example, a mean droplet diameter of 4.7 micrometers. An alternator (48) controls the vibration frequency of the ultrasonic nebulizer (45). The high-pressure jet (44) hits the coating (46) on the ultrasonic nebulizer at an angle of between 15 and 30 degrees. This minimizes the depth of the liquid layer over the ultrasonic nebulizer, which improves efficiency. This design provides several advantages:
High output, as all the liquid released from the ultrasonic nebulizer should be already aerosolized.
High efficiency, as the liquid is already in the form of small droplets, thus needing less energy for further nebulization. The ultrasound energy does not have to penetrate a long distance before being used to nebulize the liquid, as only a thin layer of the liquid covers the ultrasonic nebulizer.
A simple design, as there is no need to control the depth of the liquid layer over the nebulizer.
Easy control of the droplet size by changing the nebulizer vibration frequency.

The properties of the mist generated using the procedures described above can be more easily controlled than the properties of mists produced by other methods. The mists may be used to deliver medications to a patient's respiratory system or in a wide variety of non-medical uses, including irrigation, producing mists for extinguishing fires, industrial uses such as painting, and humidification.

The most preferred use of the mists is for rapidly changing the body temperature of a patient. The degree to which the patient is cooled or warmed will be determined by clinical considerations on a case-by-case basis. Reducing body temperature will be desirable for patients undergoing cardiac surgery or neurosurgery, as a treatment for stroke and to improve neurological outcome after resuscitation from cardiac arrest. Mists may be administered at a temperature only slightly below body temperature, e.g., at about 30° C., or, alternatively, may be administered at near-freezing temperatures. Warmed preparations may also be used and should, in general, not exceed a temperature of about 42° C. These mists will be desirable for patients suffering from hypothermia.

Any physiologically acceptable gas and liquid may be used for the creation of a mist for administration to a patient. Preferred gases are air and oxygen or a combination of the two. The preferred liquid for the generation of mist is saline. In general, the mist should comprise 90-99.5% (preferably 95-99%) gas by volume, and 0.5-10% (preferably 1-5%) liquid. Mists should be continually administered until the desired body temperature is reached as determined using standard methods well known in the art. In general, mists used for these applications should have as small a particle diameter size as possible.

All references cited herein are fully incorporated by reference. Having now fully described the invention, it will be understood by those of skill in the art that the invention may be performed within a wide and equivalent range of conditions, parameters and the like, without affecting the spirit or scope of the invention or any embodiment thereof.

1. A device for generating a mist, comprising:
(a) a high pressure pump (29) having a pump chamber (6) connected to a high pressure compartment (30) in a gas/liquid container (39);
(b) a gas reservoir (24) connected to said pump chamber by a gas pipe (11);
(c) a liquid reservoir (13) connected to said pump chamber by a liquid pipe (10);
(d) a gas/liquid container (39) comprising a high pressure compartment (30) and a low pressure compartment (40), wherein:
(i) said high pressure compartment (30) is connected to said pump chamber (6);
(ii) there are one or more orifices (20) within said high pressure compartment (30) that, when open, connect said high pressure compartment (30) with said low pressure compartment (40), wherein said orifices, when open, are 1-25 micrometers in diameter;
(iii) there is a mist pipe (22) connecting said low pressure compartment to means for releasing mist from said gas/liquid container.

2. The device of claim 1, wherein said one or more orifices (20) are formed by heating elements attached to said gas/liquid container (31).

3. The device of claim 2, wherein said high pressure compartment (30) further comprises a level sensor (15) and means for mixing the contents of said high pressure compartment.

4. The device of claim 3, further comprising a one-way valve (9) on said liquid pipe (10) and a one-way valve (8) on said gas pipe (11), wherein the opening and closing of said valve on said liquid pipe and said valve on said gas pipe is controlled by said level sensor (15).

5. The device of claim 4, further comprising a pressure gauge (16) connected to said high pressure compartment (30), wherein said high pressure pump (29) can be turned on or off in response to the reading of said pressure gauge (16).

6. The device of claim 5, wherein said high pressure pump (29) is a piston pump and is separated from said high pressure compartment (30) by a diaphragm (28).

7. The device of claim 6, wherein said low pressure compartment (40) is connected to said liquid reservoir (13) by a liquid drain pipe (26).

8. The device of claim 7, further comprising a balloon reservoir (38) connected to said gas line (11).

9. The device of claim 8, wherein said high pressure compartment (30) is connected to a pop off valve (19) and said low pressure compartment (40) is connected to a pop off valve (21).

10. The device of claim 9, further comprising a switch (31) for opening and closing said orifices (20).

11. The device of claim 1, further comprising an ultrasonic nebulizer (45) positioned in front of one or more orifices (20).

12. A method for generating a mist from a liquid, comprising loading said liquid in the device of any one of claims 1-11 and releasing said mist from said low pressure compartment (40) of said gas/liquid container (39).

13. A method for reducing the body temperature of a patient, comprising:
(a) administering a mist to said patient by ventilation, wherein:
(i) said mist is generated by the device of any one of claims 1-11;
(ii) said mist comprises a mixture of a physiologically acceptable gas and a physiologically acceptable liquid;
(iii) said mist is administered at a temperature below the body temperature of said patient; and
(b) maintaining the administration of said mist until said patient's body temperature is reduced.

14. The method claim 13, wherein said mist is administered at a temperature of 1° C.-30° C.

15. The method of claim 13, wherein said mist contains liquid droplets with an average size of no more than 2 microns in diameter.

16. The method of claim 13, wherein said mist comprises at least 80% gas by volume and no more than 20% liquid by volume.

17. The method of claim 13, wherein said gas comprises air or oxygen and said liquid comprises saline.

18. The method of claim 13, wherein the body temperature of said patient is reduced in preparation for or during cardiac or neurosurgery.

19. The method of claim 13, wherein the body temperature of said patient is reduced as a treatment for hemorrhagic shock, to prevent brain damage subsequent to a stroke or after resuscitation from cardiac arrest.

20. A method of increasing the body temperature of a patient, comprising:
(a) administering a mist to said patient by ventilation, wherein:
(i) said mist is generated by the device of any one of claims 1-11;
(ii) said mist comprises a mixture of a physiologically acceptable gas and a physiologically acceptable liquid;
(iii) said mist is administered at a temperature above the body temperature of said patient; and
(b) maintaining the administration of said mist until said patient's body temperature is increased.

21. The method of claim 20, wherein said mist is administered at a temperature of between 37° C. and 42° C.

22. The method of claim 20, wherein said mist contains liquid particles with an average size of no more than 2 microns in diameter.

23. The method of claim 20, wherein said mist is 95-99% gas by volume and 1-5% liquid by volume.

24. The method of claim 20, wherein said gas comprises air or oxygen and said liquid comprises saline.

25. The method of claim 20, wherein the body temperature of said patient is increased as a treatment for hypothermia.

26. A method for creating a mist comprising:
a) generating a gas/liquid mixture in a compartment under high pressure; and
b) extruding said gas/liquid mixture through one or more openings into an area of lower pressure so that the gas rapidly expands and thereby breaks the liquid into droplets to form said mist.
